(19) United States
(12) Patent Application Publication
(10) Pub. No.: US 2005/0153359 A1
(43) Pub. Date: Jul. 14, 2005

(54) Method for the evaluation of velvet antler

(76) Inventors: Allan L Schaefer, Lacombe Alberta (CA); James M. Suttie, Dunedin (NZ); Richard A. Lawrence, Lacombe Alberta (CA); Garry B. Desroches, Edmonton Alberta (CA); Pierre Lepage, Lacombe Alberta (CA)

Correspondence Address: GREENLEE WINNER AND SULLIVAN P C, 4875 PEARL EAST CIRCLE, SUITE 200, BOULDER, CO, 80301, US

(21) Appl. No.: 10/507,351

(22) PCT Filed: Mar. 11, 2003
(86) PCT No.: PCT/CA03/00334

(30) Foreign Application Priority Data
Mar. 11, 2002 (CA) 2 375 870

(51) Int. Cl. G01N 33/53; G06F 19/00; G01N 33/48; G01N 33/50

(52) U.S. Cl. 435/7.1; 702/19

(57) ABSTRACT

The invention provides a method and apparatus using infrared thermography for predicting an unknown value of an internal composition characteristic of velvet antler in vivo, in vitro, and/or during cooling or warming. The method involves generating a predictive model using a sample population of antlers to determine a relationship between a known input variable and a known value for the composition characteristic; and using the predictive model to predict the unknown value of the same composition characteristic in an antler not selected from the sample population. Using this value, a map can be made to identify sites and levels of the composition characteristic within the antler. The invention provides a method for predicting an internal composition characteristic of a velvet antler by obtaining an infrared thermographic image of the antler and visually ranking the image by comparison to a corresponding image of an antler with the desired composition characteristic. A method to predict maturity by comparing temperature differences between the antler's tip and base is provided.

### FIELD OF THE INVENTION

The invention relates to a method and apparatus for predicting an internal composition characteristic of interest of velvet antler.

### BACKGROUND OF THE INVENTION

Velvet antler refers to a unique regenerating tissue growing from the cranial pedicle predominantly from deer species. This tissue is composed of a variety of mineral, lipid, protein and endocrine factors (Suttie et al., 1989, 1998), and displays a very high growth or cell proliferation rate prior to maturation.

The utilization of deer co-products such as velvet antler is a valued part of Chinese traditional medicine dating back about 2000 years (Wang Shuazhi, 1993; Issacs, 1993). More recently, the unique composition and endocrine factors have been factually demonstrated in velvet antler (Suttie et al.,1998) and attention has been focused on understanding the effect of anatomical antler sites (Sunwoo et al., 1995; 1997) as well as period of growth (Suttie et al., 1989; Han and Jhon, 1994) on antler composition. Various patents and patent applications have identified biological properties of velvet antler such as an antler-based growth factor (Canadian Patent Application No. 2,132,219 to Mundy et al.); antler-derived bone growth factors (U.S. Pat. No. 5,408,041 to Mundy et al.); and a biogenic preparation from ossified deer antlers (Canadian Patent Application No. 2,201,768 to Vladimorovich).

Subjective grading or classification standards have been suggested and utilized based on the recognition that velvet antler composition, measured by factors such as ash content, will vary depending on the stage of maturity, size and weight (Haigh and Hudson, 1993). As such, the evaluation or pricing standards for velvet antler related to these subjective classifications and morphological measurement standards are typically applied. In general, there is agreement that the overall quality of the velvet antler is inversely proportional to its ash content, and that ash content is related to the stage of antler growth.

Velvet antler is unique in that it regenerates growth and nerve tissue every year. This growth rate and regeneration is indicative of unique growth factors and biological properties within the antler which are desirable in many forms of medical treatment around the world. Velvet antler grows exponentially from casting of the previous hard antler to the cessation of growth some 100-110 days later. Growth of the antler takes place at the tip, in contrast to the horns of Bovidae which elongate from the base. The dorsal surface (or top or cap) of the pedicle erodes or drops off due to the growth of new antler tissue, signalling the start or “button drop date” of a new antler growth season (Li and Suttie, 2000).

During growth of velvet antler, the least differentiated and least calcified tissue is immediately proximal to the tip. Further from the tip of the velvet antler, cartilage progressively calcifies and this calcification becomes organized into true bone. As velvet antler growth begins to slow at the end of growth, the band of bone formation gradually advances until the whole antler is composed of bone. When the antler is fully calcified, the soft furry velvet skin peels off to reveal the hard, sharp bone. The ash content of the antler is used as a measure of the amount of calcification present in the velvet antler.

Deer velvet antler is removed from the animal once each year and is typically processed by drying before export to market. The time of removal is judged by the producer to maximize velvet antler size (and hence monetary value), and minimize the degree of calcification (ash or mineral content). The producer is guided by industry set grades and price indicators. However, there is currently no way of accurately assessing ash content when the antler is still growing and no way of indirectly measuring ash content in the processed antler after removal.

An objective classification process which more accurately reflects composition is needed. Commercial operators need accurate information to enable them to judge the ‘best’ time to harvest or remove the velvet antler. The concept of ‘best’ time can be client specific, but generally should maximize the weight of the antler while minimizing calcification. This is because the calcified portion of deer velvet is unlikely to confer any human health benefit. Growth and regrowth cycles vary between animal breeds and temperate zones. The male Elk in North America regrows its antlers annually, such that antlers can be harvested every year after the animal has reached a specified age. In the prime growing phase, velvet antler can grow up to 0.5 kg per day and calcification is very rapid once the antler meets maturity—days can make a difference to the ash content. Thus, monitoring to assess optimum harvest time would be very advantageous to ensure harvest before calcification can proceed beyond a point that impacts the antler value. Although there are grading specifications based on dimensions, there is such variability among stags that this grading system is useful only for coarse grouping of similar products to facilitate sale. It would be a major advance if a producer could grade the velvet antler accurately and use this information to accurately measure optimum time of removal. In fact, an objective system may soon be demanded by the velvet antler industry. For example, market reports from Korea have suggested the adoption of a product composition validation based on ash content. Also, the evolution of global standard operating procedures for food products in general may necessitate a greater element of objectivity in product classification.

Further, when a processor/purchaser evaluates velvet antlers they are typically faced with a room full of antlers which are in a frozen state. Current practice is to perform a subjective evaluation of the antlers to estimate antler maturity and/or ash content. It would be to the advantage of both the purchaser and the producer to have an objective method of predicting velvet antler, in a whole state, for maturity and/or ash content.

Previous research in the area of velvet antlers has demonstrated that the analytical procedures of axial tomography and angiography can be used successfully to determine density gradients in velvet antler (Suttie and Fennessy, 1990). However, such tests are usually conducted on sacrificed animals or harvested antler sections, and require anesthetized animals and complicated procedures using radiopaque dyes, invasive catheterization and film developing.

Clearly, there is a need for a non-invasive, non-destructive method for evaluation and classification of velvet antler both in vivo and in vitro. Infrared thermography is a non-invasive imaging procedure involving the detection, recording, and production of an image of an animal's surface temperature or thermal patterns, using instruments which can provide immediate visual and quantitative documentation of such temperature measurements. Temperature data are then interpreted using heat loss equations and specialized computer software. Infrared thermography has numerous applications in humans and animals. In humans, infrared thermography has been used for diagnosis of tumours and cardiovascular abnormalities (Clark and Cena, 1972; U.S. Pat. No. 3,245,402 to Barnes); and blood flow related diseases or vascular retinopathies of the eye (U.S. Pat. No. 5,740,809 to Baratta). In animals, infrared thermography has been used to investigate vascular lesions in pigs and leg injuries in horses (Clark and Cena, 1972; Turner et al., 1983); to determine fat content in meat post-mortem (U.S. Pat. No. 3,877,818 to Button et al.); to detect estrous in cattle (U.S. Pat. No. 3,948,249 to Ambrosini); to determine relationships such as weight in the pens of pigs (U.S. Pat. No. 5,474,085 to Hurnik et al.); to identify live animals predisposed to producing poor meat quality (U.S. Pat. No. 5,458,418 to Jones et al.; U.S. Pat. No. 5,595,444 to Tong et al.); and to determine tissue composition characteristics (U.S. Pat. No. 6,123,451 to Schaefer and Tong).

Infrared thermography has a diversity of applications in humans and animals; however, to the inventors' knowledge, use of infrared thermography to determine compositional or maturation characteristics in a unique, regenerating tissue like velvet antler has not yet been reported. An accurate, inexpensive and non-invasive system to classify antlers according to their composition or maturation characteristics is thus most desirable.

### SUMMARY OF THE INVENTION

The invention broadly provides a method and apparatus for predicting the unknown value of an internal composition characteristic of interest of velvet antler. Infrared thermographic images are obtained from the antler in vivo or in vitro, and/or from the antler when it is subjected to a temperature change, either cooling or warming/thawing.

The invention provides a method for predicting an unknown value of an internal composition characteristic of a velvet antler of an animal, comprising the steps of:
selecting a sample population from a group of antlers;
obtaining at least one infrared thermographic image of each antler in the sample population, from at least one view, wherein the image is represented as an array of pixels providing temperature data representative of temperature information at the corresponding part of the image;
calculating a value of at least one statistical measure of the temperature data for the image, wherein the value is treated as the known input variable;
conducting an assay to obtain the known value of the composition characteristic;
determining a relationship between the known input variable and the known value of the composition characteristic, thereby generating a predictive model to predict the unknown value of the same composition characteristic in a test antler not selected from the sample population;
obtaining at least one infrared thermographic image of the test antler, from at least one view, wherein the image is represented as an array of pixels providing temperature data representative of temperature information at the corresponding part of the image;
calculating a value of at least one statistical measure of the temperature data for the image;
using the predictive model, wherein the unknown value of the composition characteristic is treated as an output variable, and the statistical measure of temperature data for the image is treated as an input variable; and
solving the predictive model to provide the value of the composition characteristic of the test antler.

In another aspect, the invention provides a method of predicting an internal composition characteristic of a velvet antler when the antler is subjected to cooling, comprising the steps of:
obtaining the image of the antler in vitro from at least one view, within a first time period after removal from the animal;
obtaining at least one second infrared thermographic image of the antler in vitro from the same view at a second time period after cooling of the antler;
calculating a value of at least one statistical measure of the temperature data for the first image and the second image, wherein the temperature data represent one or more sites within the antler;
calculating a value of a temperature change at the one or more sites within the antler;
using the predictive model, wherein the value of the temperature change is treated as an input variable; and
solving the predictive model to identify the one or more sites of high calcification and low metabolic activity within the antler.

In another aspect, the invention provides a method of predicting an internal composition characteristic of a velvet antler when the antler is subjected to warming, comprising the steps of:
obtaining the image of the antler in vitro from at least one view, within a first time period after freezing of the antler;
obtaining at least one second infrared thermographic image of the antler in vitro from the same view at a second time period after warming of the antler;
selecting one or more sites within the antler for analysis of temperature data;
calculating a value of at least one statistical measure of the temperature data at the one or more sites in the first image and the second image;
calculating a value of a temperature change at the one or more sites within the antler; and
using the predictive model, wherein the value of the temperature change is treated as an input variable; and
solving the predictive model to identify the one or more sites of high calcification and low metabolic activity within the antler.

In another aspect, the invention comprises the step of using the value of the composition characteristic to make a map of the antler, wherein the map indicates sites of high and low levels of the composition characteristic within the antler. The value of the composition characteristic is compared to a pre-determined value of the composition characteristic to determine optimal harvest timing.

In another aspect, the invention provides a method comprising the steps of using the predictive model to determine a physical volume of one or more sites of low temperature; determining the physical volume of the antler; and calculating the percentage by volume of the antler displaying the one or more sites of low temperature.

In another aspect, the invention provides a method for predicting an internal composition characteristic of a velvet antler, comprising the steps of obtaining at least one infrared thermographic image of the antler, from at least one view, wherein the image is represented as an array of pixels providing temperature data representative of temperature information at the corresponding part of the image; and scoring the image by comparing the temperature information of the image to the temperature information of a corresponding image of an antler with a known value for the composition characteristic.

In another aspect, the invention provides a method for predicting maturity of a velvet antler in vivo, comprising the steps of:
at a first time period, obtaining at least one infrared thermographic image of the tip of the antler, and at least one infrared thermographic image of the base of the antler, from at least one view, wherein each image is represented as an array of pixels providing temperature data representative of temperature information at the corresponding part of the image;
at a second time period, obtaining at least one infrared thermographic image of the tip of the antler, and at least one infrared thermographic image of the base of the antler, from the same view;
calculating a value of at least one statistical measure of the temperature data of the tip of the antler and the base of the antler, at the first time period and the second time period;
calculating a value of the temperature change of the tip of the antler and the base of the antler at the first and second time periods; and
harvesting the antler before the temperature change of the tip of the antler is equal to the temperature change of the base of the antler.

In yet another aspect, the invention further provides an apparatus for predicting an internal composition characteristic of a velvet antler comprising:
a) image acquisition means for scanning the live animal or harvested antler from at least one view to obtain at least one infrared thermographic image of the animal or antler, whereby each image is represented as an array of pixels providing temperature data representative of temperature information at the corresponding part of the image; and
b) computing and storing means for:
   i) storing each image as an array of pixels providing temperature data representative of temperature information at the corresponding part of the image;
   ii) calculating a value of at least one statistical measure of the temperature data for each thermographic image;
   iii) providing a predictive model, whereby the composition characteristic is treated as an output variable, and the statistical measure of temperature data is treated as an input variable; and
   iv) solving the predictive model to provide the value of the composition characteristic; and,
c) output means for furnishing the value of the composition characteristic for the antler.

As used herein and in the claims, the terms and phrases set out below have the meanings which follow:
“Animal” is meant to refer to species in the Cervidae family including, but not limited to, elk (Cervus elaphus manitobensis, Cervus elaphus nelsoni, Cervus elaphus roosovelti, Cervus elaphus scoticus, Cervus elaphus xanthopygus); wapiti (Cervus canadensis); chinese sika (Cervus hortulorum); japanese deer (Cervus nippon); rusa stag (Cervus tumorensis russa); sambar (Cervus unicolor); Sunda sambar (Cervus timorensis); Philippine sambar (Cervus mariannus); barasingha (Cervus duvauceli); Schomburgk's deer (Cervus schomburgki); thamin or brow-antlered deer (Cervus eldi); Thorold's deer (Cervus albirostris); moose (Alces alces); spotted deer (axis axis); marsh deer (Blastocerus dichotomus); roe deer (Capreolus capreolus); fallow deer (dama dama); Pere David's deer (Elaphurus davidianus); guemals or huemuls (Hippocamelus antisensis, H. bisulcus); brocket deer (Mazama americana, M. gouazoubira, M. rufina, M. chunyi); mule deer (Odocoileus hemionus); white-tailed deer (Odocoileus virginianus); pampas deer (Ozotoceros bezoarticus); pudus (Pudu pudu, Pudu mephistophiles); and reindeer and caribou (Rangifer tarandus).

“Antler” or “antlers” means a bony horn which grows on the head of species in the Cervidae family. Antlers are bone, but have a uniform outer cortex structure with a spongy center core, and are primarily composed of calcium hydroxyapatite with smaller amounts of calcium carbonate, calcium fluoride, magnesium phosphate, and ossein.

“Antler surface area: volume ratio” means the ratio of the total area of the antler surface to the measure of the antler quantity or capacity.

“Ash” or “ash content” means the quantity of mineral matter which remains as incombustible residue of the tested substance.

“Button drop dates” means the dates at which the antler starts to grow anew each year, as indicated by the eroding or dropping off of the dorsal surface (or top or cap) of the pedicle when the new antler emerges.

“Calcification” means a process in which the mineral calcium builds up in tissue, causing it to harden.

“Composition” or “internal composition characteristic” is meant to include, but is not limited to, moisture content, ash content, protein, fat, amino acids and growth factors of the antler.

“Delta t” means the change in temperature (° C.).

“Heat slope” means the change in infrared temperature divided by the change in time post removal from freezer.

“Infrared thermographic image” means a scan output of either or both of a visual image and corresponding temperature data. The output from infrared cameras used for infrared thermography typically provides an image comprising a plurality of pixel data points, each pixel providing a temperature data point which can be further processed by computer software to generate for example, mean temperature for the image, or a discrete area of the image, by averaging the data points over a number of pixels.

“Input variables” mean the empirical observations used in a predictive model.

“In vitro” means removed from the animal.

“In vivo” means live or on the animal.

“Measure of central tendency” means a statistical measure of a point near the centre of a group of data points. Without limitation, the term includes the mean, median and mode. The mean temperature is the most preferred measure of central tendency used in the present method. For each image, the mean temperature is determined from the average pixel temperature for a discrete area of the live animal or harvested antler which has been scanned. The mean temperature determined for each image is the arithmetic mean of the pixel temperature for the discrete area, identified as say the 70×90 pixels of the image in that discrete area.

“Measure of dispersion” is meant to include statistical measures of spread from the measure of central tendency for the group. Preferred measures of dispersion when the measure of central tendency is the mean, include variance, range, standard deviation, coefficient of variation, and standard error. Most preferred is standard deviation.

“Moisture” or “moisture content” means the loss in weight sustained by the antler or portion thereof when dried at a specific temperature under fixed conditions, and is expressed as a percentage of the weight of the original sample.

“Non-steady state” means a condition in which an animal's endocrine, physiological or metabolic values are in a state of flux often due to environmental factors such as stressors.

“Output variable” means the predictive value or hypothesized value, which is then tested empirically against actual or direct measures of outcome.

“Photoperiod” means the regularly recurring changes in the relation of light and darkness, or the rhythm of certain biological phenomena as determined by changes in the relation of light and darkness.

“Predictive model” means a predictive outcome or hypothesis which is based on an inductive process requiring empirical observations.

“Standard deviation” is the positive square root of the variance for the group, the variance being the arithmetic mean of the squares of the deviations of the individual values from their arithmetic mean.

“Steady-state” means a condition in which an animal's endocrine, physiological and metabolic values are all within a normal range and the animal is not stressed.

“Total temperature” means the mean temperature of an infrared thermographic image×image area expressed as the number of pixels (e.g., if mean temperature=20° C. and the image area=200 pixels, then total temperature=20° C.×200=4000° C.).

“Velvet” means the soft tissue covered in fur which represents the start of a new antler.

“Velvet antler” means the immature antler in its growing stage, whereby the immature antler includes velvet, and a blood filled collagenous matrix which forms the rapidly growing antler structure before it calcifies to hard bone.

### BRIEF DESCRIPTION OF THE FIGURES

FIG. 1 shows a real time image (gray tone) of a stag displaying velvet antler.

FIG. 2 shows a live antler infrared image (gray tone) of a stag (animal # 165) displaying mature velvet antler.

FIG. 3 shows a live antler infrared image (gray tone) of a stag (animal # 66) displaying immature velvet antler.

FIG. 4 shows a frozen antler infrared image (gray tone) of an immature (left, animal #66) and mature (right, animal # 165) time 0 after removal from the freezer.

FIG. 5 shows a frozen antler infrared image (gray tone) of an immature (left, animal # 66) and mature (animal # 165) time 2.45 h after removal from the freezer.

FIG. 6 shows a frozen antler infrared image (gray tone) of an immature (left, animal # 66) and mature (right, animal # 165) time 5.50 h after removal from the freezer.

FIG. 7 shows a schematic diagram of antler example (animal # 165) showing where sections of antler were taken for the in vitro ash analysis (Tables 3 and 4 of Example 1).

FIG. 8 shows an antler infrared image (gray tone) immediately following harvest (time 0).

FIG. 9 shows an antler infrared image (gray tone) one hour following harvest (time 1).

### DETAILED DESCRIPTION OF THE INVENTION

The present invention provides a method and apparatus for predicting an internal composition characteristic of velvet antler by using infrared thermography. Velvet antler pertains to the immature antler in its growing stage, whereby the immature antler includes velvet and a blood filled collagenous matrix which forms the rapidly growing antler structure before it calcifies to hard bone.

Surprisingly, the inventors have discovered that the infrared thermographic expression of an antler in the velvet or immature stage of growth is correlated with the degree of maturation, verified by the extent of inorganic mineral or ash content. In addition, the inventors have discovered that this relationship is apparent when the infrared thermographic image is obtained from the antler in vivo, in vitro, and/or when the antler is subjected to a temperature change, either cooling or warming/thawing.

The invention is beneficial in predicting the maturity and composition of velvet antler in vivo. Angiographic studies of velvet antler growth (Suttie and Fennessy, 1990) have demonstrated that vascularization is apparent and extensive in growing antler. It is also known that the extent of vascularization in a tissue usually reflects the metabolic demands or activity in the tissue (Schaefer et al., 1982). In velvet antler, a young regenerating section will display a greater degree of vascularization than a mature or so called calcified or “hardened off” section.

Without being limited to such, the inventors surmise that their method using infrared thermography works to detect the internal composition characteristics of live velvet antler due to the unique circulation system in antlers. Without limitation, the inventors believe that infrared thermographic analysis produces results indicative of the internal composition of velvet antler in vivo because areas of blood flow and areas of metabolic activity within the velvet antler tend to generate more heat than areas that are calcified. Biological events tend to have huge inefficiencies, so it was surmised that antler areas with high levels of metabolic activity, or low levels of calcification, would tend to give off higher levels of heat. Additionally, blood flow to a tissue is one of the factors that can influence the intensity of an infrared thermographic scan (Clark and Cena, 1972). Areas of the antler that are calcified tend to have lower metabolic activity, and lower, or constricted, blood flow, and thus it might be surmised that they would give off lower levels of heat. The inventors have discovered that infrared thermography can be used to detect these differences in heat levels in a meaningful way that can be correlated to the ash content, or level of calcification, of the antler.

Sources other than blood flow may also contribute to heat change and heat generation. Animals must expend energy to maintain metabolic processes consonant with life. The level of energy expenditure (i.e., energy acquired through breakdown of high energy bonds or ATP to a lower state of entrophy or ADP) required by an animal depends upon the relationship between heat production and loss. Metabolic heat production by an animal is the net result of many activities. In the non-steady state condition, additional heat production can arise from physiological stress and the catabolism of tissue, shivering thermogenesis, disease, infection and the presence of tumours. In the steady-state condition, heat production can arise from the processes of digestion, muscle activity, blood flow, protein synthesis, non-shivering thermogenesis, oxidative phosphorylation within cells, normal processes of ion or electrolyte exchange across membranes (for example, sodium pump action), and from protein synthesis involving amino acid transport. Growing antler tissue expresses considerable synthesis of new tissue (i.e., protein synthesis) which itself generates heat. The inventors have discovered that infrared thermography can be used to detect such a process, as when comparing differential heat levels in the tip compared to the base of the antler.

The inventors have thus discovered that infrared thermography is advantageous in predicting the maturity and composition of velvet antler in vivo. Further, the inventors have discovered that there is both significant within antler and across antler variation in infrared thermographic temperature for antlers scanned in vivo. The inventors have recognized that it is of considerable value to develop a system which assesses ash content while the antler is in vivo. This system allows the producer to harvest velvet antler to precise specifications, and the processor to know exactly how to trim the dried velvet antler to fulfill the market requirements. Such a system is advantageous in assessing velvet antler before it is removed from the animal.

Further, the invention is beneficial in predicting the maturity and composition of velvet antler in vitro and/or when the antler is subjected to a temperature change, either cooling or warming/thawing. An antler scanned in vitro displays significant within antler and across antler variation in infrared thermographic temperature when it is evaluated immediately following harvest while it is cooling to room temperature, and when it is evaluated while warming/thawing. The inventors have found that infrared thermography is advantageous in the assessment of velvet antler in vitro in part because the different material compositions within the velvet antler have different densities and different heat capacities. Since materials of different density and heat capacities are known to heat or cool at different rates, it was speculated that antler tissue differing in density or ash composition would also display differential infrared thermographic heat characteristics in vitro. The inventors have discovered that this differential in rate of cooling and warming/thawing is measurable using infrared thermography and surprisingly, is correlated to the ash content of the measured velvet antler. As a commercially important parameter, the ash content of the antler section can thus be predicted from an infrared thermograph. Other internal composition characteristics such as protein, fat, amino acids, growth factors or endocrine values, can also be predicted from such analysis since it is known that antler sections that vary in ash content also vary in other compounds (Sunwoo et al., 1995).

The invention thus broadly provides a method and apparatus for predicting an internal composition characteristic of a velvet antler by generating a predictive model from a sample population selected from a group of live animals or harvested antlers, and using the predictive model to predict the internal composition characteristic of a test antler of a live animal or test harvested antler not selected from the sample population.

Generating the predicative model involves selecting a sample population from a group of antlers; and obtaining at least one infrared thermographic image of each antler in the sample population, from at least one view, wherein the image is represented as an array of pixels providing temperature data representative of temperature information at the corresponding part of the image. The value (or known input variable) of at least one statistical measure of the temperature data for the image is calculated. The appropriate assay is conducted to obtain the known value of the composition characteristic; and a relationship between the known input variable and the known value of the composition characteristic is determined using statistical techniques, thereby generating the predictive model.

The predictive model is then used to predict the unknown value of the same composition characteristic in a test antler not selected from the sample population by first obtaining at least one infrared thermographic image of the antler, from at least one view, wherein the image is represented as an array of pixels providing temperature data representative of temperature information at the corresponding part of the image. A value of at least one statistical measure of the temperature data for the image is calculated. Using the predictive model, the unknown value of the composition characteristic is treated as an output variable, and the statistical measure of temperature data for the image is treated as an input variable. The predictive model is solved to provide the value of the composition characteristic of the test antler.

The invention extends to a method of predicting an internal composition characteristic of a velvet antler when the antler is subjected to cooling. The first step comprises obtaining a first infrared thermographic image of the antler in vitro from at least one view, within a first time period after removal from the animal. A second infrared thermographic image of the antler in vitro is obtained from the same view at a second time period after cooling of the antler. A value of at least one statistical measure of the temperature data for each image is calculated, with the temperature data representing one or more sites within the antler. A value of a temperature change at the one or more sites within the antler is calculated. The predictive model is then used, with the value of the temperature change treated as an input variable, and the internal composition characteristic treated as the output variable. The predictive model is then solved to identify the sites of high calcification and low metabolic activity within the antler.

Further, the invention extends to a method of predicting an internal composition characteristic of a velvet antler when the antler is subjected to warming or thawing. The first step comprises obtaining a first infrared thermographic image of the antler in vitro from at least one view, within a first time period after removal from the freezer. A second infrared thermographic image of the antler in vitro is obtained from the same view at a second time period after warming or thawing of the antler. A value of at least one statistical measure of the temperature data for each image is calculated, with the temperature data representing one or more sites within the antler. A value of a temperature change at the one or more sites within the antler is calculated. The predictive model is then used, with the value of the temperature change treated as an input variable, and the internal composition characteristic treated as the output variable. The predictive model is then solved to identify the sites of high calcification and low metabolic activity within the antler.

Further, the value of the composition characteristic obtained from solving the predictive model can be used to make a map of the antler, with the map indicating sites of high and low levels of the composition characteristic within the antler. The value of the composition characteristic is compared to a pre-determined value of the composition characteristic to determine optimal harvest timing.

The invention also provides a method comprising using the predictive model to determine a physical volume of one or more sites of low temperature; determining the physical volume of the antler; and calculating the percentage by volume of the antler displaying the one or more sites of low temperature.

Further, the invention provides a method for predicting an internal composition characteristic of a velvet antler by obtaining at least one infrared thermographic image of the antler, from at least one view, wherein the image is represented as an array of pixels providing temperature data representative of temperature information at the corresponding part of the image. The image is then scored either by computer or visually by the producer by comparing the temperature information of the image to the temperature information of a corresponding image of an antler with a known value for the desired composition characteristic, or with a pre-determined or previously known or established value of the desired composition characteristic.

The invention also provides a method for predicting maturity of a velvet antler. At a first time period, at least one infrared thermographic image is obtained of the tip of the antler, and at least one infrared thermographic image is obtained of the base of the antler. Each image is represented as an array of pixels providing temperature data representative of temperature information at the corresponding part of the image. At a second time period, at least one infrared thermographic image is obtained of the tip of the antler, and at least one infrared thermographic image is obtained of the base of the antler, from the same view. A value of at least one statistical measure of the temperature data of the tip of the antler and the base of the antler at each time period is calculated. A value of the temperature change of the tip of the antler and the base of the antler at each time period is further calculated. An antler well into or late into the harvest season displays a greater degree of calcification, and thus temperature differentiation between the tip and base would be less. With this information, the producer can monitor the temperature difference between the tip and base of the antler over time, and harvest the antler before the temperature change of the tip of the antler becomes equal to the temperature change of the base of the antler, indicating an increase in calcification. The inventors note that simply the ratio of the tip:base temperature also has utility taken at a single time.

Further, the invention extends to an apparatus for predicting an internal composition characteristic of velvet antler of a live animal or a harvested velvet antler. Such an apparatus comprises image acquisition means for scanning the animal or antler from at least one view to obtain at least one infrared thermographic image of the animal or antler. Each image is represented as an array of pixels providing temperature data representative of temperature information at the corresponding part of the image. Further, the apparatus comprises computing and storing means for storing each image as an array of pixels providing temperature data, and calculating a value of at least one statistical measure of the temperature data for each thermographic image. The computing and storing means also provides a predictive model, whereby an internal composition characteristic of the antler is treated as an output variable, and the statistical measure of temperature data is treated as an input variable. The predictive model is then solved to predict the internal composition characteristic of velvet antler of live animals or harvested velvet antlers. The apparatus may also have output means for furnishing the value of the internal composition characteristic for the antler.

Specifically, any standard, commercially available infrared thermographic camera, equipment and related computer software may be used; for example, in the present invention, the infrared camera is the Inframetrics 760 broadband camera fitted with a 0.5×lens (Inframetrics Comp. North Bellercia, Mass.), although any one of several commercially available cameras fitted with different lenses may be used to capture an infrared thermographic image, or a scan output of either or both of a visual image and corresponding temperature data. The output from infrared cameras typically provides an image comprising a plurality of pixel data points, with each pixel providing a temperature data point. Pixel values can be assigned specific color values for displaying the images visually on a computer monitor and “illustrating” the collected data, instead of using gray tones for presentation of data. Although gray tone images may be used, the inventors have used color images for Examples 1 and 2 and found color images to be more advantageous than gray tones for applications such as visual scoring of the images.

Temperature data are then further processed by computer software to generate for example, mean temperature for the image, or a discrete area of the image, by averaging the data points over a number of pixels. Suitable software for analyzing the thermographic images includes ThermaCAM™ Reporter Professional, ThermaCAM™ Explorer, ThermaCAM™ Researcher (FLIR Systems, Boston, Mass.); NIH Image and ImageJ (NIH, Washington State); Scion Image (Scion Corp., Frederick, Md.); Thermogram Image Software (Inframetrics Inc., North Bellercia, Mass.); Viewscan Software (Viewscan Ltd., Concord, ON.); and TIP Image Software (Ottawa, Canada), although any other software capable of analyzing thermographic images may be used. A suitable computer can be any modem PC-compatible computer connected to a standard monitor and a printer to furnish a hardcopy of the data and results.

The invention can be applied to species in the Cervidae family, including but not limited to, elk (Cervus elaphus manitobensis, Cervus elaphus nelsoni, Cervus elaphus roosovelti, Cervus elaphus scoticus, Cervus elaphus xanthopygus); wapiti (Cervus canadensis); chinese sika (Cervus hortulorum); japanese deer (Cervus nippon); rusa stag (Cervus timorensis russa); sambar (Cervus unicolor); Sunda sambar (Cervus timorensis); Philippine sambar (Cervus mariannus); barasingha (Cervus duvauceli); Schomburgk's deer (Cervus schomburgki); thamin or brow-antlered deer (Cervus eldi); Thorold's deer (Cervus albirostris); moose (Alces alces); spotted deer (axis axis); marsh deer (Blastocerus dichotomus); roe deer (Capreolus capreolus); fallow deer (dama dama); Pere David's deer (Elaphurus davidianus); guemals or huemuls (Hippocamelus antisensis, H. bisuicus); brocket deer (Mazama americana, M. gouazoubira, M. rufina, M. chunyi); mule deer (Odocoileus hemionus); white-tailed deer (Odocoileus virginianus); pampas deer (Ozotoceros bezoarticus); pudus (Pudu pudu, Pudu mephistophiles); and reindeer and caribou (Rangifer tarandus).

A sample population is required from a group of live animals or harvested antlers. The animals or antlers in the sample population are preferably of the same species and in sufficient numbers to provide enough data to obtain a statistically significant relationship or correlation among one or more of the selected input and output variables of interest. Such a sample size can contain as few as three animals or antlers, more preferably greater than ten animals or antlers and most preferably greater than 100 animals or antlers. The animals themselves are preferably in a steady state condition, meaning that the animal's endocrine, physiological and metabolic values are all within a normal range and the animal is not stressed. Since stressed animals may display endocrine, physiological or metabolic values which are in a state of flux due to environmental factors, such “non-steady state” animals are excluded from the sample population, since abnormal thermal expression (e.g., due to infection) may interfere with the collection of data suitable for making the predictive model of the invention. Animals can be scanned in a manner that is unobvious to the animal such as at water or feed stations. Breeds, such as elk, which are averse to capture, can be easily and readily scanned from a distance without capturing or disturbing them. Further, since the invention relates to evaluation of velvet antler, the animals are preferably in the rapid stage of antler growth as observed by the presence of velvet.

Initially, a sample population of at least three, more preferably greater than ten, and most preferably greater than 100 live animals or harvested antlers is drawn. Each animal or antler in the sample population is then scanned from a distance of approximately 1 to 3 metres using the infrared camera to generate at least one or more infrared thermographic images. The preferred distance is approximately 175-185 cm. The animal or antler can be scanned from several different views including, but not limited to, the dorsal (top), lateral (side), distal (rear), and proximal (front) views, although the dorsal (top), lateral (side) and proximal (front) views are preferred. Alternatively, representative subsections of antler can also be used.

Each obtained infrared thermographic image comprises a plurality of pixel data points (for example, typically 256 pixels wide by 207 high on a floppy disc) with each pixel providing a temperature data point representative of temperature information at the corresponding part of the image. The relative radiant surface temperature corresponding to each pixel may be represented by assigning each pixel a numerical value in the range from 0 to 255 for example. The pixel values are mapped to actual Celsius temperatures by relating them to the maximum and minimum temperature settings of the infrared camera using the following formula:

$$Actual Temperature = \frac{max temp setting - min temp setting}{256} \times pixel value$$

While much of the statistical analysis is computer-generated, pixel values can be assigned specific color values for displaying the images visually on a computer monitor and “illustrating” the collected data, instead of using gray tones for presentation of data; for example, purple may identify pixels representing temperatures less than 16° C., blue for temperatures from 16 to 19° C., and light blue for temperatures from 19 to 21° C.

Each temperature data point is then further processed by computer software to generate the value of at least one statistical measure; for example, the mean temperature for the entire image, or a portion thereof by averaging the data points over the number of pixels. Such values serve as data for each of the input variables used in the predictive model.

Statistical measures comprise measures of central tendency, dispersion, or total temperature. The measure of central tendency is selected from the mean, median, or mode or a non-parametric or rank-scale value, with the preferred measures being the mean, median and mode. The measures of dispersion can include, but are not limited to, the variance, range, standard deviation, coefficient of variation, and standard error. Total temperature relates to the mean temperature of an infrared image multiplied by the image area which is expressed in pixels.

The input variable in the present invention is thus temperature; however, other input variables, which represent animal or antler properties not derived from the infrared thermography, may be selected and included in the predictive model. Such input variables can include, but are not limited to animal weight, animal age and species type, animal genetics in the case of hybrids, antler anatomy such as length, width or circumference at specific sites, antler surface:volume ratio, button drop dates, time of year, and photoperiod. Standardized procedures for measuring these various animal or antler properties are well known to those skilled in the art.

The output variable in the present invention can include any internal composition characteristic of the antler including, but not limited to, moisture content, ash content, protein, fat, amino acids, and growth factors. Velvet antler is a complex tissue, in that the composition changes with maturity (Haines and Suttie, 2001; Mundy et al., 2001; Roubin and Ghosh, 2001; Sunwoo and Sim, 2001). Maturity is recognized as being the calcification (“hardening off”) or increase in ash content of the antler. The invention provides a method of determining this maturity stage or ash content and even vascular or non-vascular components. From predicting certain internal composition characteristics such as ash content, the relative composition of other factors (e.g., protein, fat, amino acids, growth factors) contained in the antler can also be determined. Standardized procedures for measuring any various internal composition characteristic of interest are well known to those skilled in the art. Using collected data, a relationship between the input variables and output variables is determined, thereby generating a predictive model. Any number of known statistical techniques (including, but not limited to, multiple linear regression, cluster analysis, discriminate analysis, curve fitting, ranking and artificial neural network learning, Spearman ranking, visual subjective scores) are suitable to determine such a relationship between the input and output variables.

Once the predictive model has been generated, a value for the output variable (e.g., a particular antler internal composition characteristic) can be predicted for other antlers or animals in the group of live animals or harvested antlers from which the initial sample population was drawn. A live animal or harvested antler is scanned by infrared thermography from at least one view to obtain at least one infrared thermographic image of the animal or antler, whereby each image is represented as an array of pixels providing temperature data representative of temperature information at the corresponding part of the image. The temperature data are then processed to generate a value of at least one statistical measure, wherein the value is treated as an input variable. Depending on the predictive model used, these values may be determined from the attached antler prior to removal, from the antler after removal, or both. This value and data from other input variables are entered into the predictive model, which is then solved to provide the value of the antler internal composition characteristic of interest (output variable) suitable for the nature of the commercial application.

In the present invention, the relationship is thus determined between the input variable, namely the statistical measures for the thermographic images, and the output variable, namely predicted maturity or compositional measurement. As an example, a general predictive model for predicting an internal composition characteristic of a velvet antler is thus illustrated as follows:
Y=B0+B1X1+B2X2+B3X3+B4X4+B5X5+B6X6 (1)
where:
Y=antler index or score;
B0-B6=coefficients;
X1=mean infrared thermographic image temperature (° C.) of antler;
X2=infrared thermographic image temperature (° C.) of specific antler section;
X3=body weight of animal;
X4=antler weight;
X5=species of animal;
X6=an antler anatomical feature.

The invention is thus beneficial in developing predictive models to predict internal composition characteristics of velvet antler, particularly to indicate the degree of maturity of in vivo, in vitro, cooling or warming/thawing antler tissue. As such, the invention can be used for a variety of applications, including but not limited to, the following.

In one application, a producer can determine the degree of mineralization in the growing velvet antler, and thereby accurately cut to any specification set by a prospective purchaser. The producer can then use this information to break down the antler (sometimes referred to as “stick”) in the best way possible to maximize returns from different parts of the velvet.

The producer can use infrared thermography to determine which velvet antlers among a group are likely to exhibit higher ash content. Infrared thermography can be used to map areas of higher and/or lower metabolic activity (hence, higher/lower degree of calcification) within the antler for use as a trimming tool. The areas of high calcification can be assessed and compared to overall antler area to predict the percent ash content and/or assign the antler a grade rating.

The producer can also determine the degree of maturation of the antler by monitoring the temperature difference at the tip and base of the antler over time. An antler well into or late into the harvest season displays a greater degree of calcification, and thus temperature difference between the tip and base would be less. Such knowledge can thus be beneficial in determining or predicting optimal harvest times for specific market specifications.

Since the tips of the tines and upper parts of the velvet antler are sites of active growth, they are typically desired commercially for high value products. The invention can be applied to delineate these high value areas and facilitate efficient trimming of the velvet to allocate the high value portions to appropriate markets. This aim can be achieved by relating temperature bands to levels of calcification in a map which serves as a guide to cut the velvet stick after processing.

Further, the producer can assess a desired internal composition characteristic of a velvet antler by scanning the antler to obtain one or more thermographic images of the antler. The producer can then score the antler using a computer or visually by comparing the antler's infrared thermographic image to a corresponding image of another antler with a known value for the desired maturity and composition, thereby assessing the velvet antler before harvesting. For this application, pixel values are preferably assigned specific color values for “illustrating” the collected data, instead of using gray tones.

The producer can also scan the animals frequently to determine the best cutting time, with the computer software programmed to treat ash content as the output variable. Additionally, the computer software can provide the actual cutting decision; for example, when a particular pattern is detected, a notification on the screen indicates to the producer that the animal displays a particular internal composition characteristic.

In another application, the processor can provide to a farmer an infrared camera with computer software which indicates when the velvet antler meets the required specifications for the processor's desired application. The farmer can then provide the processor with the velvet antler and its corresponding infrared thermographic image indicating the ash content.

Lastly, the infrared thermographic images can serve as a culling tool to identify and select for breeding purposes those animals which produce a high quality and quantity of velvet antler. Predictive models for specific deer breeds or species using representative sample populations of animals or antlers, and selection criteria can be developed for specific market demands.

The invention is further illustrated by the following non-limiting examples.

### Example 1

### Determination of Maturation Stage of Antler Using Infrared Thermography

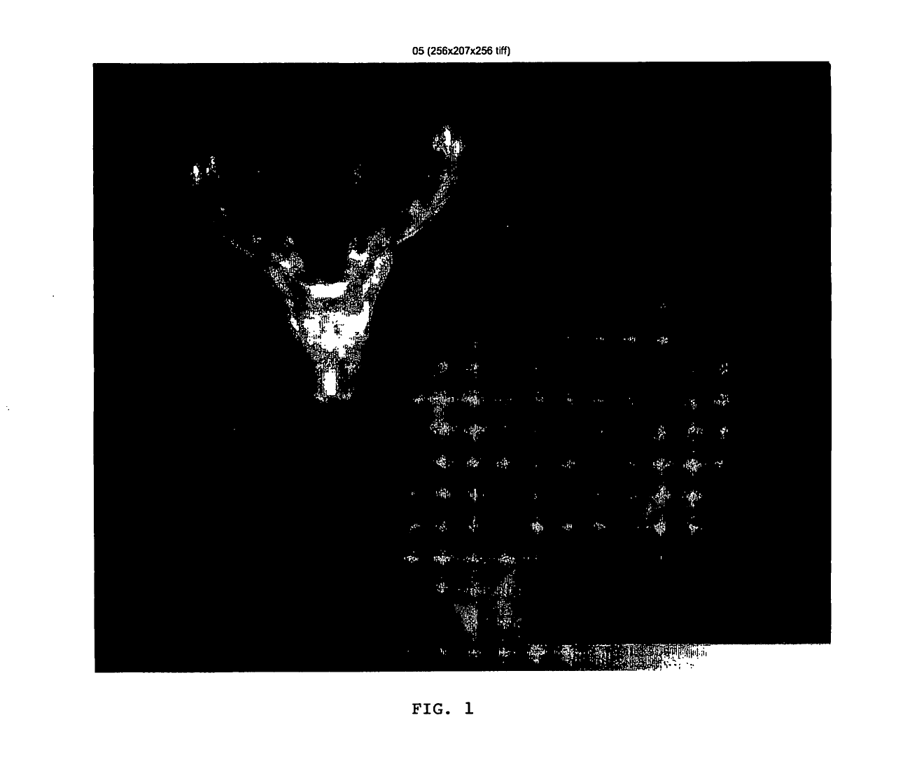

Twenty-six domestic Wapiti stags (Cervis elaphus) were used in this study conducted at the Agriculture and Agri-Food Canada Lacombe Research Centre (Lacombe, Alberta, Canada). Care was taken to maintain the animals in a steady state in the handling area. All animals were in the stage of rapid antler growth, as indicated by the presence of velvet on the left and right antlers. FIG. 1 shows a real time image (gray tone) of a stag displaying velvet antler.

The left and right antlers of all animals were scanned from a dorsal view at a distance of approximately 3 meter using a 760 Inframetrics broad band camera fitted with a 0.5× lens (Inframetrics Comp. North Bellercia, Mass.). TIP image software (Ottawa, Canada) was used for the subsequent analysis of the thermographic images.

Table 1 indicates the individual temperatures (° C.) of both left and right antlers of all animals. Specifically, the area of the image obtained from scanning is represented by the number of pixels. The minimum and maximum temperatures, mean temperature and standard deviation are presented. The results indicate that there is considerable variation in the temperature within an individual antler; for example, for animal #85, the temperature ranges from 16.4 to 37.9 within the right antler, while the temperature ranges from 17.3 to 37.4 within the left antler. There is also considerable variation in the temperature among antlers. For the right antler, the temperature ranges from 16.4 (animal #85) to 37.9° C (animal #85), while for the left antler, the temperature ranges from 17.3 (animal #85) to 37.6 (animal #43).

TABLE 1
Individual temperatures of left and right velvet antlers of Wapiti stag

| | Velvet | | Minimum | Maximum | Mean | |
|---|---|---|---|---|---|---|
| | antler | Number | temperature | temperature | temperature | Standard |
| Animal # | scanned | of pixels | (° C.) | (° C.) | (° C.) | deviation |
| 8 | right | 2954 | 26.5 | 36.7 | 32.7 | 1.3 |
| | left | 2821 | 25.6 | 35.4 | 32.2 | 1.3 |
| 20 | right | 3924 | 19.7 | 35.4 | 29.0 | 2.3 |
| | left | 3968 | 23.4 | 36.3 | 29.5 | 2.1 |
| 24 | right | 3742 | 25.6 | 36.4 | 33.1 | 1.5 |
| | left | 3688 | 27.1 | 36.2 | 33.1 | 1.3 |
| 38 | right | 3293 | 22.2 | 33.5 | 28.4 | 1.5 |
| | left | 2712 | 21.6 | 35.5 | 28.8 | 2.1 |
| 43 | right | 2645 | 19.6 | 36.7 | 32.1 | 2.0 |
| | left | 3513 | 24.5 | 37.6 | 32.5 | 1.8 |
| 52 | right | 1515 | 23.5 | 34.7 | 30.4 | 1.8 |
| | left | 1428 | 21.4 | 35.6 | 30.2 | 2.8 |
| 53 | right | 1652 | 23.3 | 33.9 | 29.6 | 1.7 |
| | left | 1934 | 20.5 | 33.7 | 28.7 | 1.7 |
| 54 | right | 1835 | 22.4 | 35.3 | 29.6 | 1.7 |
| | left | 1749 | 23.9 | 35.1 | 29.5 | 1.6 |
| 59 | right | 1796 | 21.4 | 35.0 | 29.9 | 2.0 |
| | left | 1870 | 20.4 | 34.4 | 30.1 | 2.1 |
| 63 | right | 1559 | 22.6 | 34.8 | 29.4 | 1.8 |
| | left | 1412 | 24.4 | 33.2 | 29.3 | 1.7 |
| 64 | right | 1772 | 23.9 | 35.5 | 29.7 | 2.0 |
| | left | 1919 | 21.4 | 34.7 | 29.8 | 1.8 |
| 66 | right | 1666 | 23.6 | 35.0 | 30.4 | 1.6 |
| | left | 1507 | 22.1 | 34.4 | 30.0 | 1.5 |
| 67 | right | 2439 | 20.9 | 36.3 | 30.7 | 2.2 |
| | left | 2686 | 23.8 | 35.6 | 30.5 | 1.7 |
| 69 | right | 3448 | 22.3 | 35.5 | 30.9 | 1.5 |
| | left | 3503 | 23.5 | 36.0 | 30.4 | 1.7 |
| 70 | right | 2297 | 22.5 | 35.5 | 28.9 | 1.7 |
| | left | 2722 | 22.0 | 36.1 | 28.5 | 2.0 |
| 71 | right | 2238 | 26.5 | 35.2 | 31.9 | 1.8 |
| | left | 2246 | 24.1 | 34.6 | 31.5 | 1.3 |
| 72 | right | 2695 | 23.2 | 34.2 | 28.2 | 2.1 |
| | left | 3162 | 20.0 | 35.8 | 28.7 | 2.6 |
| 78 | right | 4465 | 20.5 | 34.7 | 29.4 | 1.8 |
| | left | 4049 | 23.6 | 34.5 | 29.4 | 1.7 |
| 85* | right | 3309 | 16.4 | 37.9 | 29.6 | 3.1 |
| | left | 2882 | 17.3 | 37.4 | 29.3 | 3.0 |
| 88 | right | 1967 | 22.4 | 36.9 | 30.1 | 1.8 |
| | left | 1782 | 24.6 | 34.5 | 30.4 | 1.8 |
| 90 | right | 3645 | 24.8 | 35.9 | 32.7 | 1.4 |
| | left | 3700 | 23.5 | 37.2 | 32.8 | 1.0 |
| 91 | right | 3233 | 26.0 | 35.9 | 32.7 | 1.2 |
| | left | 3641 | 22.5 | 36.6 | 32.8 | 1.4 |
| 103 | right | 2324 | 22.4 | 35.7 | 30.7 | 1.7 |
| | left | 2179 | 21.1 | 35.0 | 29.5 | 1.7 |
| 131 | right | 2816 | 21.9 | 35.1 | 29.1 | 1.6 |
| | left | 2970 | 21.5 | 34.5 | 29.6 | 1.6 |
| 165* | right | 3025 | 18.8 | 35.2 | 29.7 | 2.9 |
| | left | 2915 | 23.1 | 34.5 | 30.4 | 1.7 |
| Mean | | 2622 | 22.7 | 35.4 | 30.3 | 1.8 |
| Range** | right | | 16.4 | 37.9 | | |
| | left | | 17.3 | 37.6 | | |

*Animals #85 and #165 displayed calcification
**Range within antlers (−2.8° C. to 1.9° C. or 4.7° C.) from mean

Table 2 indicates the combined temperature (° C.) of the left and right antlers of all animals. Specifically, the area of the image obtained from scanning is represented by the number of pixels. The minimum and maximum temperatures, mean temperature, standard deviation, and range within the antlers are presented.

TABLE 2
Combined temperature of left and right velvet antlers of Wapiti stag

| | | Maximum | Minimum | Mean | | Range |
|---|---|---|---|---|---|---|
| | Number | temperature | temperature | temperature | Standard | within |
| Animal # | of pixels | (° C.) | (° C.) | (° C.) | deviation | antlers |
| 8 | 2887.5 | 26.1 | 36.1 | 32.4 | 1.3 | −2.1 |
| 20 | 3946.0 | 21.5 | 35.9 | 29.2 | 2.2 | 1.1 |
| 24 | 3715.0 | 26.4 | 36.3 | 33.1 | 1.4 | −2.8 |
| 38 | 3002.5 | 21.9 | 34.5 | 28.6 | 1.8 | 1.7 |
| 43 | 3079.0 | 22.0 | 37.1 | 32.3 | 1.9 | −2.0 |
| 52 | 1562.0 | 24.7 | 35.1 | 30.2 | 2.3 | 0.2 |
| 53 | 1793.0 | 21.9 | 33.8 | 29.2 | 1.7 | 1.2 |
| 54 | 1792.0 | 23.2 | 35.2 | 29.6 | 1.7 | 0.7 |
| 59 | 1833.0 | 20.9 | 34.7 | 30.0 | 2.1 | 0.3 |
| 63 | 1485.5 | 23.5 | 34.0 | 29.3 | 1.8 | 1.0 |
| 64 | 1845.5 | 22.6 | 35.1 | 29.7 | 1.9 | 0.6 |
| 66 | 1586.5 | 22.8 | 34.7 | 30.2 | 1.6 | 0.1 |
| 67 | 2562.5 | 22.4 | 35.9 | 30.6 | 1.9 | −0.3 |
| 69 | 3475.5 | 23.0 | 35.7 | 30.7 | 1.6 | −0.3 |
| 70 | 2509.5 | 22.3 | 35.8 | 28.7 | 1.8 | 1.6 |
| 71 | 2242 | 25.3 | 34.9 | 31.7 | 1.5 | −1.4 |
| 72 | 2928.5 | 21.6 | 35.0 | 28.4 | 2.3 | 1.9 |
| 78 | 4257.0 | 22.0 | 34.6 | 29.4 | 1.7 | 0.9 |
| 85 | 3095.5 | 16.8 | 37.7 | 29.5 | 3.1 | 0.9 |
| 88 | 1874.5 | 23.5 | 35.7 | 30.2 | 1.8 | 0.1 |
| 90 | 3672.5 | 24.1 | 36.6 | 32.7 | 1.2 | −2.4 |
| 91 | 3437.0 | 24.3 | 36.2 | 32.8 | 1.3 | −2.4 |
| 103 | 2251.5 | 21.8 | 35.3 | 30.1 | 1.7 | 0.2 |
| 131 | 2893.0 | 21.7 | 34.8 | 29.4 | 1.6 | 1.0 |
| 165 | 2970.0 | 21.0 | 34.8 | 30.0 | 2.3 | 0.3 |
| Mean | 2622.0 | 22.7 | 35.4 | 30.3 | 1.8 | |

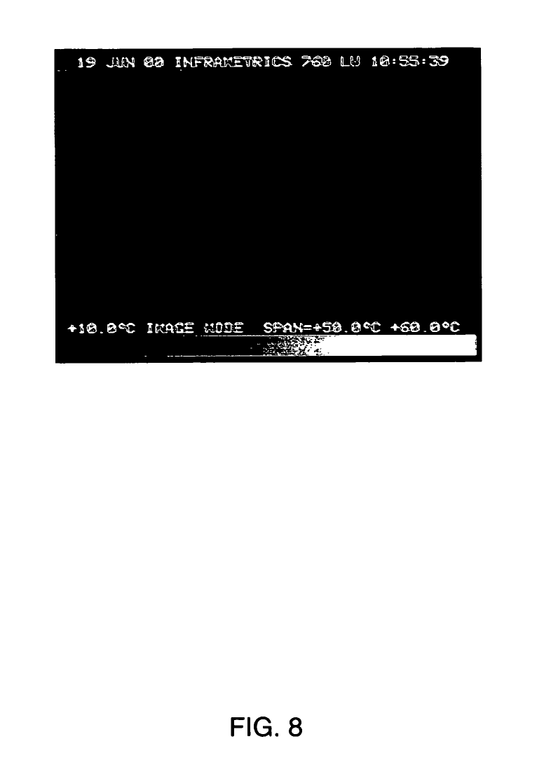

The left and right antlers were removed from all animals using standard methods known to those skilled in the art (Canadian Food Inspection Agency, 1998). The antlers were then frozen to −20° C. Since materials of different density and heat capacities are known to heat or cool at different rates, it is reasonable that antler tissue differing in density or ash composition also displays differential infrared thermographic heat characteristics in vitro. Antler from animal #165, as shown in FIGS. 8 to 13, was removed from a −20° C. freezer and placed at room temperature to rethaw for 5 hours (20° C.), with a circulating fan used to maintain a constant temperature over the entire antler. The antler was scanned while rethawing using a 760 Inframetrics broad band camera fitted with a 0.5× lens (Inframetrics Comp. North Bellercia, Mass.). Continuous data tape collection of data was possible, however, infrared thermographic images were captured at 0 hour, 2.45 hours and 5 hours post removal from the freezer. The differential rate of antler section thawing was recorded. The thermographic scan changes were seen to display different rates (slope) of heating suggesting that the assessment or classification of the antler could also be conducted on thawing, or cooling, antler tissue in vitro.

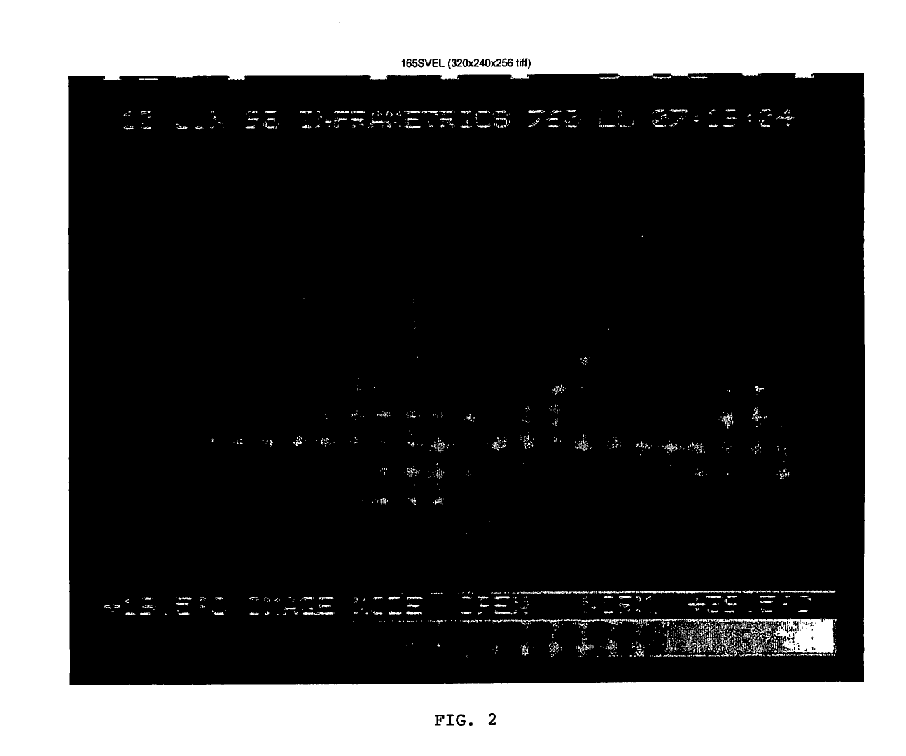

FIGS. 2-6 illustrate the variation in temperature and further illustrate that for the most metabolically active tissue, an increased temperature on live tissue or heating slope on thawing tissue is evident whether measured in the live tissue or in the thawing antler. The lighter areas of shade equate to warmer temperatures. Pixel values were assigned specific color values for “illustrating” the collected data, instead of using gray tones for presentation of data. Although gray tone images may be used, the inventors used color images for Example 1 and found color images to be more advantageous than gray tones.

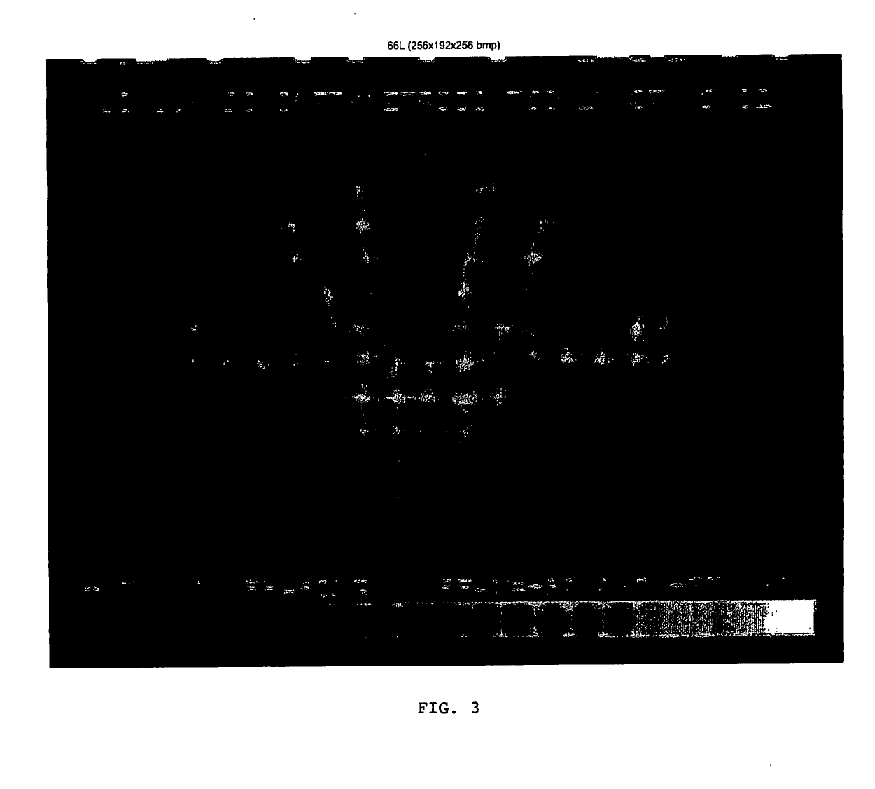

FIG. 2 shows a live antler infrared (gray tone) image of a stag (animal # 165) displaying mature velvet antler, while FIG. 3 shows a live antler infrared (gray tone) image of a stag (animal # 66) displaying an immature velvet antler. The image of the immature velvet antler displays lighter tones compared to the image of the mature velvet antler which shows darker tones. The temperature of immature, actively growing velvet antler is thus warmer than mature, more calcified velvet.

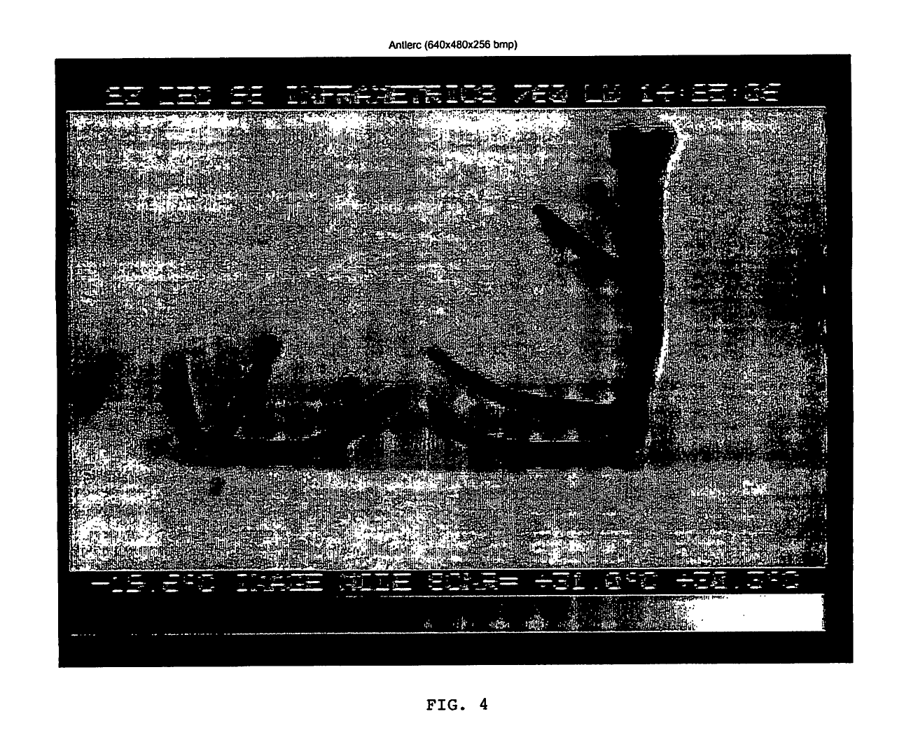

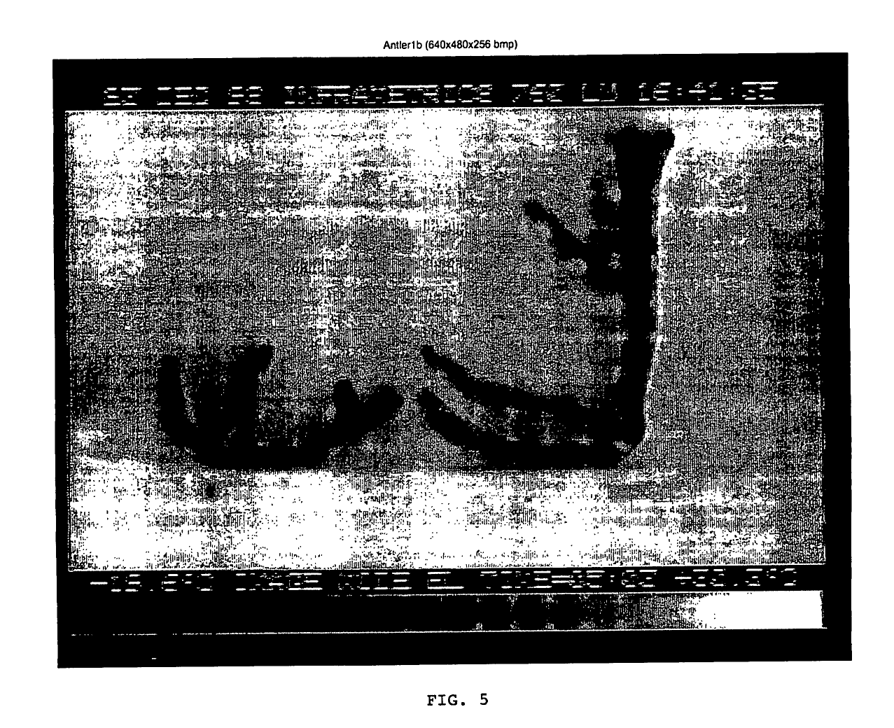

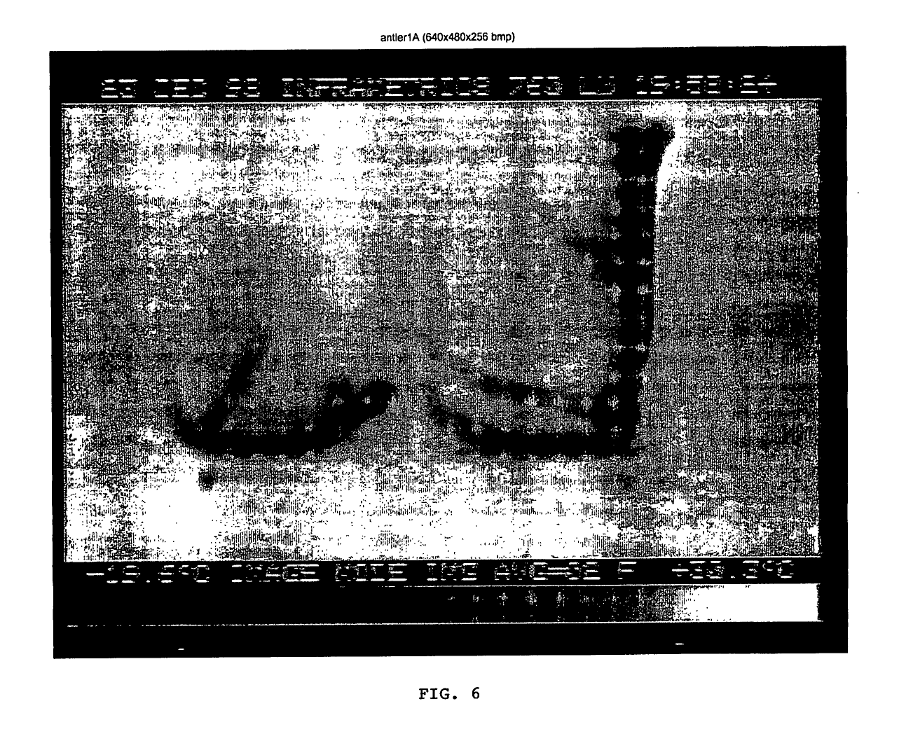

FIGS. 4-6 compare immature and mature frozen antlers at times 0 hours, 2.45 hours and 5 hours post-removal from the freezer. The gray tones in the images of the immature and mature frozen antlers become lighter as the antlers warm or thaw; however, the image of the immature antler displays lighter tones compared to the image of the mature antler which shows darker tones. FIG. 4 shows a frozen antler infrared image (gray tone) of an immature (left, animal # 66) and mature (right, animal # 165) time 0 h after removal from the freezer, while FIG. 5 shows a frozen antler infrared image (gray tone) if an immature (left, animal # 66) and mature (animal # 165) time 2.45 h after removal from the freezer. FIG. 6 shows a frozen antler infrared image (gray tone) of an immature (left, animal # 66) and mature (right, animal # 165) time 5.5 h after removal from the freezer. Throughout the rethawing, the temperature of immature, actively growing velvet antler remained warmer than mature, more calcified velvet. More calcified velvet antler is of lesser value to a producer due to its higher mineral content. Thus, velvet antler of higher overall temperature is likely to reflect lower ash content and hence, be of higher value to a producer.

Representative samples from these antlers were subsequently analyzed for moisture and ash contents using standard methods (AOAC, 1995). Table 3 shows the moisture and ash contents of specific sections of antler of animal #165. Table 4 shows the moisture and ash contents, temperature of the antler of the live animal, and the heat slope from animal #165. The most metabolically active tissue displays the lowest ash content.

Antlers were selected from animal #165 which displayed a mature rack of velvet antler. This animal was monitored in June 1998 and was from a domestic herd of wapiti stags in central Alberta. To facilitate data collection, the animal was brought from outdoor paddocks, with pen mates, into an enclosed handling facility designed specifically for wapiti. The animal and the velvet antler were scanned while unrestrained soon after arrival into the facility. A 760 Inframetrics broad band infrared camera was used to scan the animal at approximately 3 meters. The animal was subsequently restrained in a holding facility and the velvet antler removed using standard methods known to those skilled in the art (Canadian Food Inspection Agency, 1998). The removed antler was subsequently frozen and then scanned after later removal from the freezer and thawing.

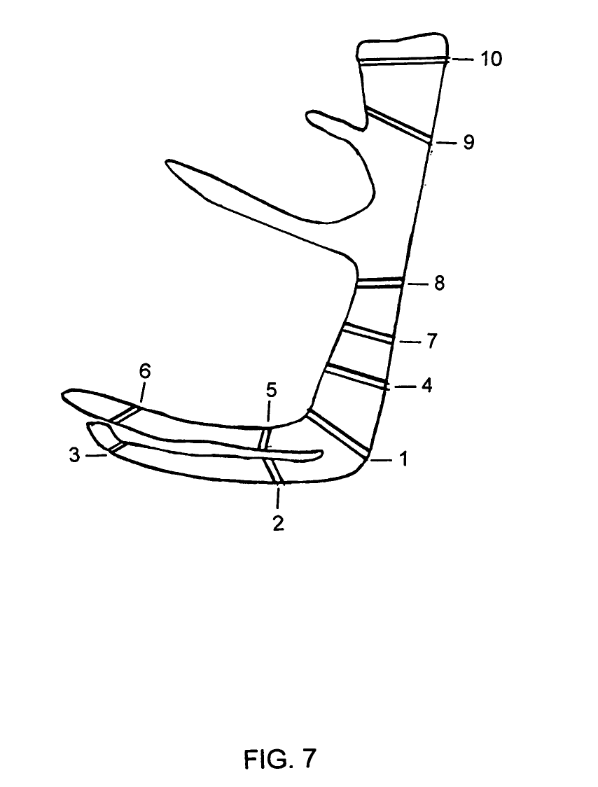

For compositional analysis, selective slices of 0.5 cm thickness were sectioned from a specific antler (animal # 165), as illustrated schematically in FIG. 7 which identifies the sections of antler which were selected for chemical ash analysis (Tables 3 and 4). These sections were dried to constant weight and subsequently analyzed for ash content using conventional procedures (AOAC, 1995). Briefly, the antler sections were placed in a muffle oven held at approximately 550° C. for 40 h and the samples then measured gravimetrically. The following information was collected on the antler: live infrared thermographic temperature profile, infrared thermographic profile from the thawing antler at different times post removal from the freezer, and ash analysis of the antler from selective anatomical sites. Interestingly, the correlation coefficient between the in vivo and/or in vitro antler temperature profile collected from the identified sites and the ash composition was apparent, suggesting a close or high degree of relationship between infrared thermographic profile and objective antler composition.

TABLE 3
Moisture and ash contents of antler sections from animal #165

| Antler Section # | Moisture (%) | Ash (%) |
|---|---|---|
| 1 | 47.6 | 24.0 |
| 2 | 55.1 | 17.9 |
| 3 | 65.6 | 9.2 |
| 4 | 51.0 | 21.1 |
| 5 | 50.4 | 21.9 |
| 6 | 63.1 | 11.0 |
| 7 | 53.4 | 18.9 |
| 8 | 62.1 | 12.0 |
| 9 | 63.1 | 12.0 |
| 10 | 81.1 | 1.4 |

TABLE 4
Moisture and ash contents, temperature of live antler and heat slope from
animal #165

| | | | Live Antler | | | |
|---|---|---|---|---|---|---|
| Antler | Moisture | Ash | Temperature | Heat Slope* | Heat Slope* | Heat Slope |
| Section # | (%) | (%) | (° C.) | at time 0-2.45 h | at time 2.45-5.00 h | (mean) |
| 1 | 47.6 | 24.0 | 29.2 | 4.4 | 2.4 | 3.4 |
| 8 | 62.1 | 12.0 | 29.9 | 4.9 | 1.2 | 3.0 |
| 10 | 81.1 | 1.4 | 31.0 | 4.6 | 2.0 | 3.3 |

*heat slope = change in infrared temperature/change in time post removal from freezer
**The correlation coefficient between ash content and infrared thermal data was −0.9867 and between moisture and infrared thermal data was 0.9987.

### Example 2

Fourteen domestic wapiti stags (Cervis elaphus) raised at an Alberta Game farm, and used in this study conducted at the Agriculture and Agri-Food Canada Lacombe Research Centre (Lacombe, Alberta, Canada). Care was taken to maintain the animals in a steady state in the handling area. All animals were in the stage of rapid antler growth, as indicated by the presence of velvet on the antlers and the display of higher temperatures at the tip of the antler compared to the temperature at the base of the antler. The antlers were removed from all animals using standard methods known to those skilled in the art (Canadian Food Inspection Agency, 1998).

The antlers were scanned in vivo and after harvest to record the infrared thermographic images as the antler cooled. The antlers of all animals were scanned using a 760 Inframetrics broad band camera fitted with a 0.5× lens (Inframetrics Comp. North Bellercia, Mass.). TIP image software (Ottawa, Canada) was used for the subsequent analysis of the thermographic images. The antlers were scanned at time 0 (immediately when the antler was removed from the animal) and at time 1 (approximately 1 hour from time 0). The term “delta t” represents the temperature change (° C.) per hour.

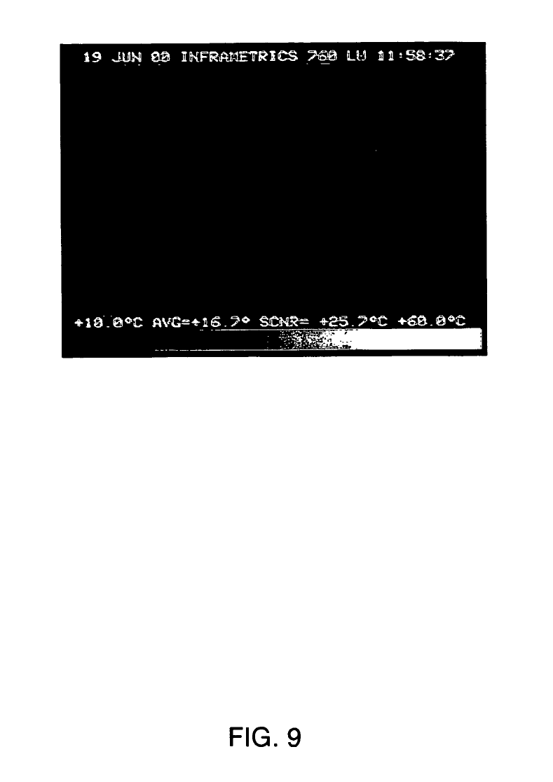

Pixel values were assigned specific color values for “illustrating” the collected data, instead of using gray tones for presentation of data. Although gray tone images may be used, the inventors used color images for Example 2. FIG. 8 shows an antler infrared image (gray tone) immediately following harvest (time 0). FIG. 9 shows an antler infrared image (gray tone) one hour following harvest (time 1).

Table 5 indicates the base and tip temperatures of all animals at times 0 and 1, and the change in temperature. When the antlers were removed and allowed to cool at room temperature (approximately 25° C.) for the duration of the time period (1 hour), the tip (less calcified) and the base (more calcified) display differential cooling rates. The change in temperature for the tip was 4.55° C. per hour compared to 3.79° C. per hour for the base (P=0.02).

It has been reported that the tip and base have different compositional differences (Li and Suttie, 2001); thus, at least part of the differential cooling reflects such compositional differences. Infrared thermographic images, which are obtained on antlers within a reasonable period following their harvest, can thus be used to predict antler maturity and/or ash content. Further, cooling rates for antler at different stages of maturity would likely vary; for example, an antler well into or late into the harvest season displays a greater degree of calcification, and thus temperature differentiation between the tip and base would be less. Such knowledge can thus be beneficial in determining or predicting optimal harvest times for specific market specifications.

TABLE 5
Comparison of Antler Base and Tip Temperatures of Wapiti Stag

| Animal | | | | Tip | | |
|---|---|---|---|---|---|---|
| identification | Base | | | Time | Time | Delta |
| number | Time 0 | Time 1 | Delta T | 0 | 1 | T |
| 13f | 29.1 | 25.5 | 3.3 | 30.4 | 25.3 | 4.6 |
| 10f | 27.3 | 23.3 | 5.2 | 28.5 | 25.2 | 4.3 |
| 45y | 27.9 | 23.7 | 4.2 | 28.6 | 23.8 | 4.7 |
| 25f | 30.8 | 28.1 | 2.4 | 31.9 | 27.7 | 3.6 |
| 9726y | 31.6 | 27.8 | 3.3 | 32.4 | 26.3 | 5.2 |
| 128b | 26.6 | 21.9 | 4.4 | 29.9 | 23.5 | 6.0 |
| 129b | 25.7 | 21.2 | 4.3 | 26.4 | 22.2 | 4.0 |
| 143b | 27.0 | 21.9 | 4.7 | 28.8 | 23.3 | 5.1 |
| 139b | 29.7 | 25.3 | 4.3 | 29.8 | 27.2 | 2.6 |
| 141b | 30.0 | 26.8 | 2.6 | 31.5 | 25.9 | 4.7 |
| 142b | 30.4 | 26.1 | 3.9 | 31.6 | 26.2 | 4.5 |
| 3hp | 29.8 | 25.7 | 3.6 | 30.7 | 25.8 | 4.3 |
| 74hb | 30.9 | 26.8 | 3.7 | 31.8 | 25.8 | 5.4 |
| 157g | 29.0 | 25.7 | 3.2 | 29.6 | 24.7 | 4.8 |
| Mean | 29.0 | 25.0 | 3.8 | 30.1 | 25.2 | 4.6 |
| Standard | 1.8 | 2.2 | 0.8 | 1.7 | 1.6 | 0.8 |
| deviation | | | | | | |
| t value base vs. | 0 | 0.47 | 0.024 | | | |
| tip | | | | | | |
| | P < 0.01 | P > 0.05 | P = 0.02 | | | |

### References

Association of Official Analytical Chemists. 1995. Official Methods of Analysis, AOCS. International. 16^{th} ed. Washington, D.C.
Canadian Food Inspection Agency (1998) Code of Practice for Farmed Deer (Cervidae). Animal Health and Protection Division. Ottawa, Ontario.
Clark, J. A. and Cena, K. (1972) Thermographic measurements of the surface temperatures of animals. J. Mammol. 54:1003-1007.
Drew, K. R. (1990) Venison and other deer products. Proceedings, 2^{nd} Int. Deer Biology Conference. Mississippi.
Haigh, J. C. and Hudson, R. J. (1993) Farming Wapiti and Red Deer. Mosby-Year Book Inc., pp. 150-152.
Haines, S. R. and Suttie, J. M. (2001) Near-infrared spectroscopy for antler composition analysis. In J. S. Sim, H. H. Sunwoo, R. J. Hudson and B. T. Jeon. 2001. Antler Science and Product Technology. Antler Science and Product Technology Research Centre, Edmonton, Canada (ISBN 1-896110-14-2), pp. 135-150.
Han, N. Y. and Jhon, G. A. (1994) Purification and analysis of gangliosides from deer antlers. Kov. Biochem J. 27:459-465.
Issacs, C. (1993) Velvet usage. In. A Salute to World Deer Farming. Proceedings of the 1^{st} World Deer Congress, Christchurch, N. Z. New Zealand Deer Farmers Association. Wellington, N. Z., pp. 205-206.
Li, C. and Suttie, J. M. (2001) Deer Antler Generation: A Process from Permanent to Deciduous. In J. S. Sim, H. H. Sunwoo, R. J. Hudson and B. T. Jeon. (eds) 2001. Antler Science and Product Technology. Antler Science and Product Technology Research Centre, Edmonton, Canada (ISBN 1-896110-14-2), pp. 15-31.
Mundy, G. R., Gutierrez, G., Gallwitz, W., Feng, J., Chen, D., Garrett, R. and Harris, S. (2001) Antler derived bone growth factors and their potential for use in osteoporosis. In J. S. Sim, H. H. Sunwoo, R. J. Hudson and B. T. Jeon. (eds) 2001. Antler Science and Product Technology. Antler Science and Product Technology Research Centre, Edmonton, Canada (ISBN 1-896110-14-2), pp. 171-187.
Roubin, R. And Ghosh, P. (2001) Deer antler cartilage cells express specific growth factors which up-regulate chondrocyte DNA and proteoglycan biosynthesis in vitro. In J. S. Sim, H. H. Sunwoo, R. J. Hudson and B. T. Jeon. (eds) 2001. Antler Science and Product Technology. Antler Science and Product Technology Research Centre, Edmonton, Canada (ISBN 1-896110-14-2), pp. 151-170.
Schaefer, A. L., Young, B. A. and Turner, B. V. (1982) The effect of cold exposure on blood flow distribution in sheep. J. Thermal Biol. 7:15-21.
Sunwoo, H. H. and Sim, J. S. (2001) Morphological, chemical and molecular characteristics of active components in velvet antlers for biomedicine and nutraceuticals. In J. S. Sim, H. H. Sunwoo, R. J. Hudson and B. T. Jeon. (eds) 2001. Antler Science and Product Technology. Antler Science and Product Technology Research Centre, Edmonton, Canada (ISBN 1-896110-14-2), pp. 111-134.
Suttie, J. M.; Fennessy, P. F.; Corson, I. D.; Laas, F. J.; Crosbie, S. F.; Butler, J. H. and Gluckman, P. D. (1989) Pulsatile growth hormone, insulin-like growth factors and antler development in red deer (Cervus alaphus scoticus) stags. J. Endocrin. 121:351-360.
Suttie, J. M. and Fennessy, P. F. (1990) Antler regeneration studies with antler removal, axial tomography and angiography. In. Horns, Pronghorns and Antlers. Springer-Verlag.

N.Y., pp. 313-338.
Suttie, J. M.; Li, C.; Bubenik, G. A. and Rolf, H. J. (1998) Studies of antler growth: a review of literature. Advances in Deer Biology. Procs. 4^{th} International Deer Biology Congress, Kaposvar, Tipo Express Ltd. Kaposvar, pp. 375-382.
Sunwoo, H. H.; Nakano, T.; Hudson, R. J. and Sim, J. S. (1995) Chemical composition of antlers from wapiti (Cervus elaphus). J. Agric. Food Chem. 43:2846-2849.
Sunwoo, H. H.; Sim, L. Y. M.; Nakano, T.; Hudson, R. J. and Sim, J. S. (1997) Glycosaminoglycans from growing antlers of wapiti (Cervus elaphus) Can. J. Anim.

Sci. 77:715-721.
Turner, T. A.; Fessler, J. F.; Lamp, M.; Pearce, J. A. and Geddes, L. A. (1983) Thermographic evaluation of horses with podotrochlosis. Am. J. Vet. Res. 44: 535-539.
Wang Shuazhi, W. (1 993) The utilization of deer co-products in China. Procs. of the First World Deer Farming Congress, Christchurch, New Zealand, pp. 209-210.
Wu, Y. (1992) Solid state NMR study of bone mineral. PHD Thesis, MIT.
Zetti-Schaffer, K. F., Ghaffarpour, M., McGovern, T. F. and Engh, C. A. (1993) Scanning electron microscope of bone growth into 40% and 80% porous coated AML prosthesis retrieved at autopsy. 39^{th} Annual meeting, Orthopedic Research Society 18:471.

### Patent Literature

Ambrosini, H. J. Method and apparatus for detecting and identifying a cow in heat. U.S. Pat. No. 3,948,249, issued Apr. 6, 1976.
Baratta, F. I. Noninvasive infrared blood flow detector. U.S. Pat. No. 5,740,809, issued Apr. 21, 1998.
Barnes, R. B. Process of diagnosis by infrared thermography. U.S. Pat. No. 3,245,402, issued Apr. 12, 1966.
Button, G. F. and Norris, K. H. Photo-optical method for determining fat composition in meat. U.S. Pat. No. 3,877,818, issued Apr. 15, 1975.
Glimcher, M. J.; Kim, H. M. and Rey, C. Isolation of the calcium-phosphate crystals of bone. U.S. Pat. No. 5,691,397, issued Nov. 25, 1997.
Hurnik, D.; Ireland, W. P.; Stahlbaum, B. W.; Dawson, W. E. Remote thermographic sensing of livestock. U.S. Pat. No. 5,474,085, issued Dec. 12, 1995.
Jones, S. D. M.; Schaefer, A. L.; Tong, A. K. W.; Scott, S. L.; Gariepy, C. D. J. and Graham, R. C. Method for detecting poor meat quality in live animals. U.S. Pat. No. 5,458,418, issued Oct. 17, 1995.
Mundy, G. R.; Gutierrez, G. E.; Garrett, I. R.; Sabatini, M.; Crumley, G. R.; Morse, C. C.; Arnett, T. R.; Izbicka, E. and Burgess, W. Novel antler derived bone growth factors. Canadian Patent Application No. 2,132,219, published Sep. 30, 1993.
Mundy, G. R.; Gutierrez, G. E.; Garrett, I. R.; Sabatini, M.; Izbicka, E.; Burgess, W.; Crumley, G. R.; Morse, C. C. and Arnett, T. R. Process of purifying antler-derived bone growth factors. U.S. Pat. No.5,408,041, issued Apr. 18, 1995.
Schaefer, A. L. and Tong, A. K. W. Process for determining a tissue composition characteristic of an animal. U.S. Pat. No. 6,123,451, issued Sep. 26, 2000.
Tong, A. K. W., Jones, S. D. M. and Schaefer, A. L. Method for detecting poor meat quality in groups of live animals. U.S. Pat. No. 5,595,444, issued Jan. 21, 1997.
Vladimirovich, T. V. Powdery biogenic preparation from ossified deer antlers and a food additive based on said biogenic preparation. Canadian Patent Application No. 2,201,768, published Oct. 11, 1997.

All publications mentioned in this specification are indicative of the level of skill in the art to which this invention pertains. All publications are herein incorporated by reference to the same extent as if each individual publication was specifically and individually indicated to be incorporated by reference.

Although the foregoing invention has been described in some detail by way of illustration and example, for purposes of clarity and understanding it will be understood that certain changes and modifications may be made without departing from the scope or spirit of the invention as defined by the following claims.

1. A method for predicting an unknown value of an internal composition characteristic of a velvet antler of an animal, comprising the steps of:
selecting a sample population from a group of antlers;
obtaining at least one infrared thermographic image of each antler in the sample population, from at least one view, wherein the image is represented as an array of pixels providing temperature data representative of temperature information at the corresponding part of the image;
calculating a value of at least one statistical measure of the temperature data for the image, wherein the value is treated as the known input variable;
conducting an assay to obtain the known value of the composition characteristic;
determining a relationship between the known input variable and the known value of the composition characteristic, thereby generating a predictive model to predict the unknown value of the same composition characteristic in a test antler not selected from the sample population;
obtaining at least one infrared thermographic image of the test antler, from at least one view, wherein the image is represented as an array of pixels providing temperature data representative of temperature information at the corresponding part of the image;
calculating a value of at least one statistical measure of the temperature data for the image;
using the predictive model, wherein the unknown value of the composition characteristic is treated as an output variable, and the statistical measure of temperature data for the image is treated as an input variable; and
solving the predictive model to provide the value of the composition characteristic of the test antler.

2. The method of claim 1, wherein the composition characteristic is selected from the group consisting of moisture content, ash content, protein, fat, amino acids, growth factors, and location and amount of calcification.

3. The method of claim 1, wherein the composition characteristic is ash content.

4. The method of claim 2, further comprising the step of including in the predictive model, one or more input variables not derived from infrared thermography.

5. The method of claim 4, wherein the one or more input variables are selected from the group consisting of animal weight, animal age, species type, genetic breed, antler length, antler width, antler circumference, antler geometric measure, antler surface to volume ratio, button drop dates, time of year, and photoperiod.

6. The method of claim 5, wherein the animal is selected from the group consisting of Cervus elaphus manitobensis, Cervus elaphus nelsoni, Cervus elaphus roosovelti, Cervus elaphus scoticus, Cervus elaphus xanthopygus, Cervus canadensis, Cervus hortulorum, Cervus nippon, Cervus timorensis russa, Cervus unicolor, Cervus timorensis, Cervus mariannus, Cervus duvauceli, Cervus schomburgki, Cervus eldi, Cervus albirostris, Alces alces, axis axis, Blastocerus dichotomus, Capreolus capreolus, dama dama, Elaphurus davidianus, Hippocamelus antisensis, H. bisulcus, Mazama americana, M. gouazoubira, M. rufina, M. chunyi, Odocoileus hemionus, Odocoileus virginianus, Ozotoceros bezoarticus, Pudupudu, Pudu mephistophiles, and Rangifer tarandus.

7. The method of claim 6, wherein the view of the image is a dorsal, lateral, distal, or proximal view.

8. The method of claim 7, wherein the statistical measure is selected from the group consisting of a measure of central tendency, a measure of dispersion, and a total temperature.

9. The method of claim 7, wherein the statistical measure is the mean temperature.

10. The method of claim 8, wherein the relationship between the known input variable and the known value for the composition characteristic is determined by a statistical technique selected from the group consisting of multiple linear regression, cluster analysis, discriminate analysis, curve fitting, ranking and artificial neural network learning, Spearman ranking, and visual subjective scores.

11. The method of claim 10, wherein the image is obtained from the antler in vivo or in vitro.

12. The method of claim 10, wherein the image is obtained from the antler in vivo or in vitro and when subjected to a temperature change.

13. The method of claim 12, wherein the temperature change is cooling.

14. The method of claim 12, wherein the temperature change is warming.

15. The method of claim 13, further comprising the steps of:
obtaining the image of the antler in vitro from at least one view, within a first time period after removal from the animal;
obtaining at least one second infrared thermographic image of the antler in vitro from the same view at a second time period after cooling of the antler;
calculating a value of at least one statistical measure of the temperature data for the first image and the second image, wherein the temperature data represent one or more sites within the antler;
calculating a value of a temperature change at the one or more sites within the antler;
using the predictive model, wherein the value of the temperature change is treated as an input variable; and
solving the predictive model to identify the one or more sites of high calcification and low metabolic activity within the antler.

16. The method of claim 14, further comprising the steps of:
obtaining the image of the antler in vitro from at least one view, within a first time period after freezing of the antler;
obtaining at least one second infrared thermographic image of the antler in vitro from the same view at a second time period after warming of the antler;
selecting one or more sites within the antler for analysis of temperature data;
calculating a value of at least one statistical measure of the temperature data at the one or more sites in the first image and the second image;
calculating a value of a temperature change at the one or more sites within the antler; and
using the predictive model, wherein the value of the temperature change is treated as an input variable; and
solving the predictive model to identify the one or more sites of high calcification and low metabolic activity within the antler.

17. The method of claim 11, further comprising the step of using the value of the composition characteristic to make a map of the antler, wherein the map indicates sites of high and low levels of the composition characteristic within the antler.

18. The method of claim 11, further comprising the step of comparing the value of the composition characteristic to a pre-determined value of the composition characteristic to determine optimal harvest timing.

19. The method of claim 18, wherein the composition characteristic is ash content.

20. The method of claim 11, further comprising the steps of:
using the predictive model to determine a physical volume of one or more sites of low temperature;
determining the physical volume of the antler; and
calculating the percentage by volume of the antler displaying the one or more sites of low temperature.

21. A method for predicting an internal composition characteristic of a velvet antler, comprising the steps of:
obtaining at least one infrared thermographic image of the antler, from at least one view, wherein the image is represented as an array of pixels providing temperature data representative of temperature information at the corresponding part of the image; and
scoring the image by comparing the temperature information of the image to the temperature information of a corresponding image of an antler with a known value for the composition characteristic.

22. The method of claim 21, wherein scoring is conducted visually or by computing means.

23. The method of claim 22, wherein the composition characteristic is ash content.

24. The method of claim 23, wherein the animal is selected from the group consisting of Cervus elaphus manitobensis, Cervus elaphus nelsoni, Cervus elaphus roosovelti, Cervus elaphus scoticus, Cervus elaphus xanthopygus, Cervus canadensis, Cervus hortulorum, Cervus nippon, Cervus timorensis russa, Cervus unicolor, Cervus timorensis, Cervus mariannus, Cervus duvauceli, Cervus schomburgki, Cervus eldi, Cervus albirostris, Alces alces, axis axis, Blastocerus dichotomus, Capreolus capreolus, dama dama, Elaphurus davidianus, Hippocamelus antisensis, H. bisulcus, Mazama americana, M. gouazoubira, M. rufina, M. chunyi, Odocoileus hemionus, Odocoileus virginianus, Ozotoceros bezoarticus, Pudu pudu, Pudu mephistophiles, and Rangifer tarandus.

25. The method of claim 24, wherein the view of the image is a dorsal, lateral, distal, or proximal view

26. A method for predicting maturity of a velvet antler in vivo, comprising the steps of:
at a first time period, obtaining at least one infrared thermographic image of the tip of the antler, and at least one infrared thermographic image of the base of the antler, from at least one view, wherein each image is represented as an array of pixels providing temperature data representative of temperature information at the corresponding part of the image;
at a second time period, obtaining at least one infrared thermographic image of the tip of the antler, and at least one infrared thermographic image of the base of the antler, from the same view;
calculating a value of at least one statistical measure of the temperature data of the tip of the antler and the base of the antler, at the first time period and the second time period;
calculating a value of the temperature change of the tip of the antler and the base of the antler at the first and second time periods; and
harvesting the antler before the temperature change of the tip of the antler is equal to the temperature change of the base of the antler.

27. The method of claim 26, wherein the animal is selected from the group consisting of Cervus elaphus manitobensis, Cervus elaphus nelsoni, Cervus elaphus roosovelti, Cervus elaphus scoticus, Cervus elaphus xanthopygus, Cervus canadensis, Cervus hortulorum, Cervus nippon, Cervus timorensis russa, Cervus unicolor, Cervus timorensis, Cervus mariannus, Cervus duvauceli, Cervus schomburgki, Cervus eldi, Cervus albirostris, Alces alces, axis axis, Blastocerus dichotomus, Capreolus capreolus, dama dama, Elaphurus davidianus, Hippocamelus antisensis, H. bisulcus, Mazama americana, M. gouazoubira, M. rufina, M. chunyi, Odocoileus hemionus, Odocoileus virginianus, Ozotoceros bezoarticus, Pudu pudu, Pudu mephistophiles, and Rangifer tarandus.

28. The method of claim 27, wherein the view of the image is a dorsal, lateral, distal, or proximal view

29. The method of claim 28, wherein the statistical measure is selected from the group consisting of a measure of central tendency, a measure of dispersion, and a total temperature.

30. The method of claim 28, wherein the statistical measure is the mean temperature.

31. The method of claim 29, wherein the image is obtained from the antler in vivo or in vitro.

32. The method of claim 29, wherein the image is obtained from the antler in vivo or in vitro and when subjected to a temperature change.

33. The method of claim 32, wherein the temperature change is cooling.

34. The method of claim 32, wherein the temperature change is warming.

35. An apparatus for predicting an internal composition characteristic of a velvet antler comprising:
a) image acquisition means for scanning the live animal or harvested antler from at least one view to obtain at least one infrared thermographic image of the animal or antler, whereby each image is represented as an array of pixels providing temperature data representative of temperature information at the corresponding part of the image; and
b) computing and storing means for:
i) storing each image as an array of pixels providing temperature data representative of temperature information at the corresponding part of the image;
ii) calculating a value of at least one statistical measure of the temperature data for each thermographic image;
iii) providing a predictive model, whereby the composition characteristic is treated as an output variable, and the statistical measure of temperature data is treated as an input variable; and
iv) solving the predictive model to provide the value of the composition characteristic; and,
c) output means for furnishing the value of the composition characteristic for the antler.

36. The apparatus of claim 35, wherein the composition characteristic is selected from the group consisting of moisture content, ash content, protein, fat, amino acids, growth factors, and location and amount of calcification.

37. The apparatus of claim 35, wherein the composition characteristic is ash content.

38. The apparatus of claim 36, further comprising one or more input variables not derived from infrared thermography.

39. The apparatus of claim 38, wherein the one or more input variables are selected from the group consisting of animal weight, animal age, species type, genetic breed, antler length, antler width, antler circumference, antler geometric measure, antler surface to volume ratio, button drop dates, time of year, and photoperiod.

40. The apparatus of claim 38, wherein the statistical measure is selected from the group consisting of a measure of central tendency, a measure of dispersion, and a total temperature.

41. The apparatus of claim 38, wherein the statistical measure is the mean temperature.
